(19) United States
(12) Patent Application Publication
(10) Pub. No.: US 2013/0085454 A1
(43) Pub. Date: Apr. 4, 2013

(54) REVERSIBLE DISPOSABLE RESIN NEEDLE FOR BLOOD VESSEL

(71) Applicants: Gyeong-Jin Song, Jeonju-si (KR); Yeong-Sik Song, Seoul (KR); Won-Gi Song, Seoul (KR); Heon Chae, Seoul (KR); Jung-Sik Song, Seoul (KR); In-Jeong Song, Seoul (KR); Je-Wan Chae, Seoul (KR); Won-Chan Song, Seoul (KR)

(72) Inventors: Gyeong-Jin Song, Jeonju-si (KR); Yeong-Sik Song, Seoul (KR); Won-Gi Song, Seoul (KR); Heon Chae, Seoul (KR); Jung-Sik Song, Seoul (KR); In-Jeong Song, Seoul (KR); Je-Wan Chae, Seoul (KR); Won-Chan Song, Seoul (KR)

(21) Appl. No.: 13/702,670

(22) PCT Filed: Mar. 10, 2011
(86) PCT No.: PCT/KR11/01660

§ 371 Date: Dec. 7, 2012

(30) Foreign Application Priority Data
Jun. 18, 2010 (KR) 10-2010-0058057

(51) Int. Cl. A61M 5/315

(52) U.S. Cl. CPC A61M 5/31505
USPC 604/220

(57) ABSTRACT

Provided is a reversible disposable resin needle for blood vessels. The reversible disposable resin needle for blood vessels is decreased in the total length compared with conventional resin needles while preventing exposure to the outside of a cylinder tube which forms the outer container when a piston tube consisting of a metallic needle is pushed back, and thus can reduce manufacturing costs and improve the convenience of storage, distribution, and handling.

### TECHNICAL FIELD

The present invention relates to a resin needle for a blood vessel, which includes a resin tube and a metallic needle used to inject blood or an injectable solution such as an aqueous solution into a blood vessel, and more particularly, to a reversible and disposable resin needle for a blood vessel, which is decreased in a total length compared with a conventional resin needle while preventing exposure to an outer container when a piston tube consisting of the metallic needle is pushed back, and thus can reduce manufacturing costs and improve convenience of storage, distribution, and handling.

### BCKGROUND ART

A resin needle for a blood vessel that is generally used is configured so that injection into a blood vessel is performed using a metallic needle, which is inserted in a resin tube while a leading edge thereof partially protrudes, and then the metallic needle is separated from the resin tube and a fluid tube is connected to the resin tube to supply the blood or aqueous solution. However, after inserting the resin tube into the blood vessel using the metallic needle, the metallic needle should be instantly removed in order to connect a blood tube or a fluid tube, and the blood or the aqueous solution leaks out of the resin tube inserted in the blood vessel while performing the connection. In addition, when the fluid tube is connected, there is a risk of infection such as acquired immune deficiency syndrome (AIDS), hepatitis, or another pathogen. In particular, when the blood or the aqueous solution is injected into a patient without knowing that the patient has AIDS, a person handling the metallic needle that is removed from the blood vessel of the patient and contains the blood of the patient is likely to cause infection if he/she is pricked by the metallic needle.

In order to resolve the above problem, the present applicant has registered Korean Registered Patent Registration No. 0616246 regarding an improved reversible disposable resin needle for a blood vessel; however, the above invention has a complicated structure and expensive production costs, and thus is not suitable for mass production and it is difficult for it to become widely distributed. Moreover, a handle of the resin needle should be rotated repeatedly, and thus it is inconvenient to use the resin needle. To address the above problem, the present applicant has suggested an improved reversible disposable resin needle for a blood vessel in Korean Patent Application No. 10-2008-0006485. However, the resin needles for blood vessels that have been filed hitherto by the present applicant mostly have a structure in which a piston tube that is an inner body is slidably inserted in a cylinder tube that is an outer body, and thus the injection solution (blood or an aqueous solution) infiltrates a gap between the piston tube and the cylinder tube or the blood in the blood vessel may flow back. Also, the conventional resin needles have a structure connecting the cylinder tube and the piston tube using a film cylinder that is formed of a thin film material so as to prevent the blood or the aqueous solution from leaking and prevent impurities from infiltrating from the outside; however, such a resin needle has a complex structure and thus is difficult to fabricate. In particular, the film cylinder formed of a thin film is easily damaged because it is exposed to the outside, and moreover, if an operator does not recognize damage of the film cylinder, a serious medical accident may occur.

### DISCLOSURE

### Technical Problem

The present invention is directed to a reversible and disposable resin needle for a blood vessel, which has an improved structure of a piston tube and a cylinder tube to be easily handled while increasing a flowing amount of an aqueous solution, to have a decreased length compared to the conventional art to improve convenience in usage and reduce fabrication costs with high air-tightness, and moreover, to prevent a medical accident due to reckless handling in advance by restricting back and forth positions of a piston moved in the cylinder tube.

### Technical Solution

One aspect of the present invention provides a reversible disposable resin needle for blood vessels including: a cylinder tube including a body having a resin tube and an escape prevention portion; a piston tube including a metallic needle, a connecting tube in which a fluid tube is inserted, and a separation prevention protrusion blocked by the escape prevention portion; and a gasket that is a watertight element in slidable contact with an inner surface of the cylinder tube while in close contact with the inner surface, wherein the piston tube has a length of about 60 to 90% based on an entire length of the cylinder tube, the resin tube has a length of about 40 to 60 mm, and a gap between the inner surface of the resin tube and an outer surface of the metallic needle is about 0.05 to 1 mm.

Another aspect of the present invention provides a reversible disposable resin needle for blood vessels including: a cylinder tube including a body having a resin tube and an escape prevention portion; a piston tube including a metallic needle, a connecting tube in which a fluid tube is inserted, and a separation prevention protrusion blocked by the escape prevention portion; and a film cylinder that is a sealing element of a cylindrical shape having opposite ends respectively connected to a rear inner surface of the body of the cylinder tube and an outer end surface of the connecting tube at a rear portion of a gasket of the piston tube, wherein the piston tube has a length of about 60 to 90% based on an entire length of the cylinder tube, the resin tube has a length of about 40 to 60 mm, and a gap between the inner surface of the resin tube and an outer surface of the metallic needle is about 0.5 to 2 mm.

Another aspect of the present invention provides a reversible disposable resin needle for blood vessels including: a cylinder tube including a body having a resin tube and an escape prevention portion; a piston tube including a metallic needle in which a solution transfer path is formed, a connecting tube in which a fluid tube is inserted and including a gasket in close contact with an inner surface of the cylinder tube on an outer surface of the piston tube, and a separation prevention protrusion blocked by the escape prevention portion; and a film cylinder that is a sealing element of a cylindrical shape having opposite ends respectively connected to a rear inner surface of the body of the cylinder tube and an outer end surface of the connecting tube at a rear portion of a gasket of the piston tube, wherein the piston tube has a length of about 60 to 90% based on an entire length of the cylinder tube, the resin tube has a length of about 40 to 60 mm, and a gap between the inner surface of the resin tube and an outer surface of the metallic needle is about 0.5 to 2 mm.

Another aspect of the present invention provides a reversible disposable resin needle for blood vessels including: a cylinder tube including a body having a resin tube and an escape prevention portion; a piston tube, in which a fluid tube is inserted, including a metallic needle, in which a solution transfer path is formed, and a separation prevention protrusion blocked by the escape prevention portion; and a gasket integrally formed on an end portion of a connection portion of the piston tube so that an outer surface thereof may be in slidable contact with the inner surface of the cylinder tube while in close contact therewith, wherein the piston tube has a length of about 60 to 90% based on an entire length of the cylinder tube, the resin tube has a length of about 40 to 60 mm, and a gap between the inner surface of the resin tube and an outer surface of the metallic needle is about 0. 5 to 2 mm.

### Advantageous Effects

According to a reversible disposable resin needle for blood vessels of the present invention, when blood or an aqueous solution is injected, after inserting a metallic needle in a blood vessel, the metallic needle may be retrieved by a simple manipulation. Thus, the reversible disposable resin needle for blood vessels of the present invention is easily handled, and since the retrieval operation of the metallic needle is made accurately, the convenience of usage may be improved. Also, mass production and reduction of manufacturing costs can be expected due to a simple structure. Moreover, the resin needle is discarded in a state in which the metallic needle is received in a cylinder tube, and thus secondary infection of AIDS, typhoid, syphilis, and hepatitis caused by reckless handling of a user can be prevented, and infection of a patient due to external pathogens during the injection can be prevented. In particular, since lengths of the cylinder tube and a piston tube are shorter than those of the conventional art, it is easy to handle, store, and distribute the resin needles, and the manufacturing costs may be reduced. Also, since a body length of the resin needle is reduced, there is a low risk of the needle falling out of the blood vessel even when the patient moves his/her joints, arms or body. Thus, the patient may move freely while receiving the injection.

### DESCRIPTION OF DRAWINGS

FIGS. 1 through 6 are diagrams showing a reversible disposable resin needle for a blood vessel according to a first embodiment of the present invention;

FIGS. 7 through 12 are diagrams showing a reversible disposable resin needle for a blood vessel according to a second embodiment of the present invention;

FIGS. 13 through 18 are perspective views showing a reversible disposable resin needle for a blood vessel according to a third embodiment of the present invention;

FIGS. 19 through 24 are perspective views showing a reversible disposable resin needle for a blood vessel according to a fourth embodiment of the present invention; and

FIGS. 24 through 28 are diagrams showing a reversible disposable resin needle for a blood vessel according to a fifth embodiment of the present invention.

### DESCRIPTION OF MAJOR SYMBOL IN THE ABOVE FIGURE

| | |
|---|---|
| 1: resin needle for blood vessel | 10: cylinder tube |
| 20: piston tube | 22: solution transfer path |
| 25: connection tube portion | 30: film cylinder |

### Mode for Invention

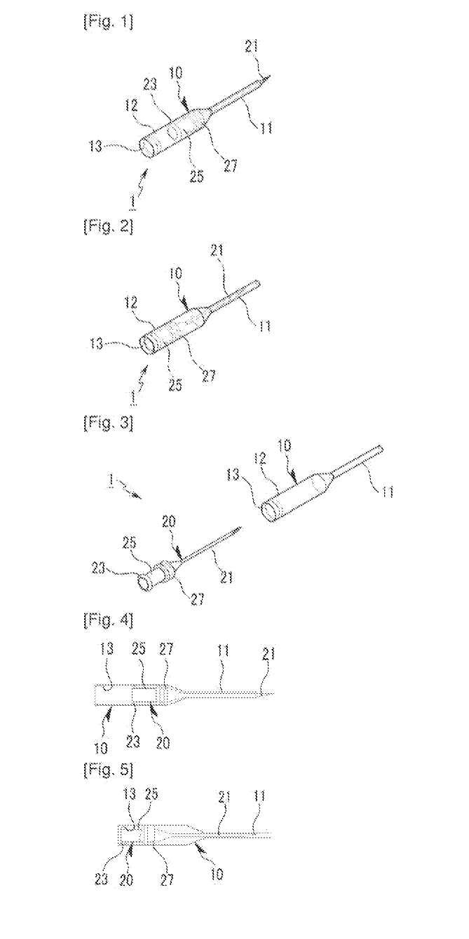

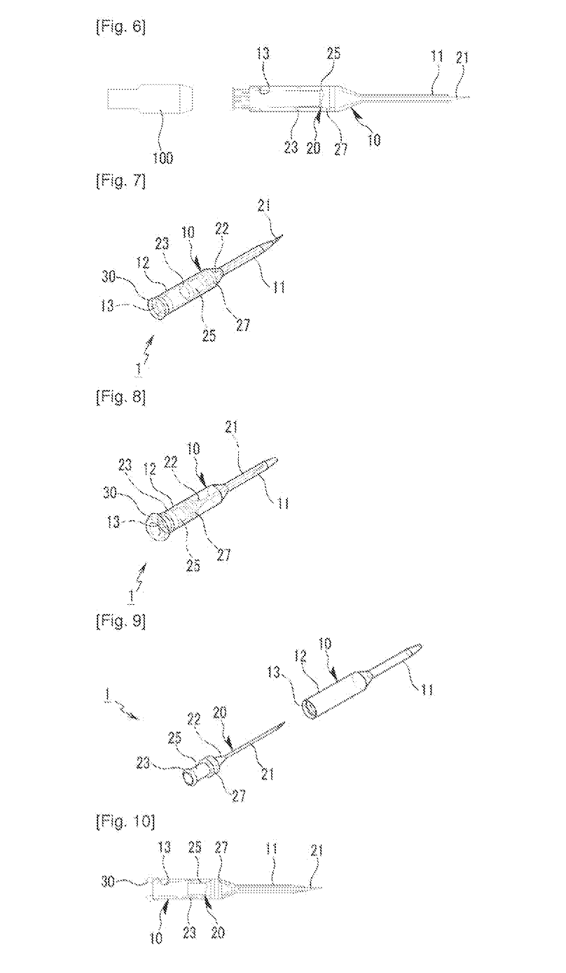

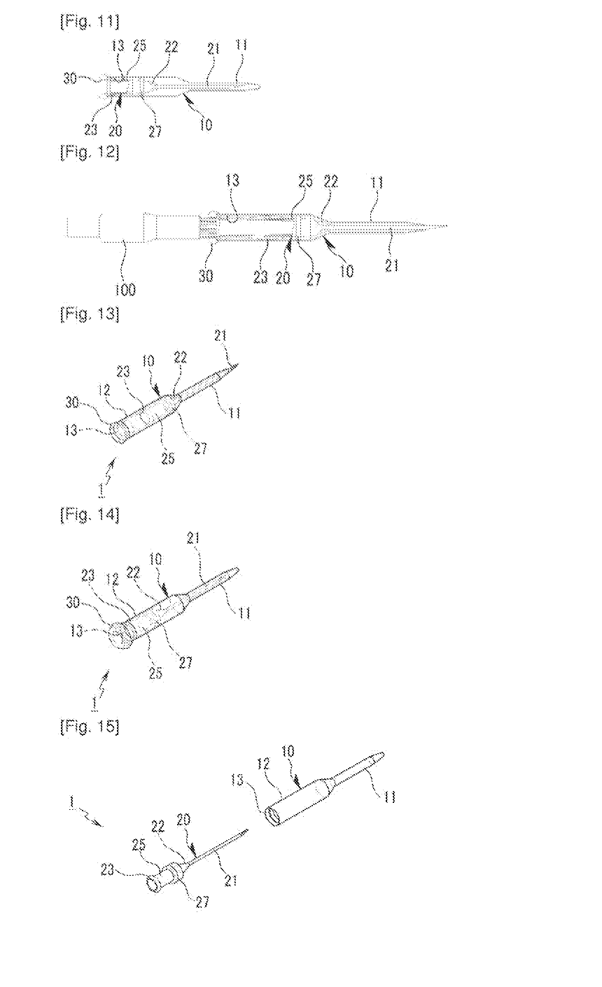

The present invention will now be described more fully with reference to the accompanying drawings, in which exemplary embodiments of the invention are shown. Like numbers refer to like elements throughout. In the description of the present invention, if it is determined that a detailed description of commonly-used technologies or structures related to the invention may unnecessarily obscure the subject matter of the invention, the detailed description will be omitted. FIGS. 1 through 6 are diagrams showing a reversible disposable resin needle for a blood vessel according to an embodiment of the present invention. FIG. 1 shows a state in which a piston tube 20 moves forward in a cylinder tube 10 so that a metallic needle 21 is exposed through a leading edge of a resin tube 11, FIG. 2 shows a state in which the piston tube 20 of FIG. 1 is retrieved, and FIG. 3 is an exploded perspective view of the cylinder tube 10 and the piston tube 20 separating from each other. FIGS. 4 and 5 are cross-sectional views showing internal structures of the reversible disposable resin needle for a blood vessel of FIG. 1, and FIG. 6 is a cross-sectional view showing a state in which a fluid tube is connected to the reversible disposable resin needle for a blood vessel of FIG. 1.

The reversible disposable resin needle for a blood vessel according to the present embodiment is connected to a well known solution set (not shown) for injecting blood or an injection solution such as an aqueous solution into a blood vessel. The reversible disposable resin needle for a blood vessel includes the cylinder tube 10 formed of a flexible and transparent material, the piston tube 20 configured to be inserted into the cylinder tube 10 to slidably move back and forth selectively according to an operation of an operator, and a gasket 27 for sealing the leading edge of the piston tube 20 and an inside of the cylinder tube 10 from each other.

The cylinder tube 10 is a member having a tubular shape body 12 formed of a transparent or semitransparent and hard or flexible material, and has a leading edge from which a thin and long resin tube 11 integrally protrudes and an opened rear end. Also, an escape prevention portion 13 that is a kind of projection for preventing the piston tube 20 to be described later from separating during movement to a retrieval position protrudes from a rear inner surface of the body 12 of the resin tube 11. That is, the cylinder tube 10 of the present embodiment is an element configuring an outer body of a syringe while receiving the solution or the blood to guide the solution or the blood to be injected into a blood vessel of a patient through the resin tube 11, and thus is formed as a hollow cylinder shape. Preferably, the cylinder tube 10 is formed of a transparent or semitransparent material so that an injection state of the injection solution (an aqueous solution or blood) can be easily checked from the outside, and may be molded using a hard or soft material for convenience of handling.

The piston tube 20 is a member moving back and forth in the cylinder tube 10. The piston tube 20 includes the metallic needle 21, which has a hollow inner portion and partially exposes through the leading edge of the resin tube 11 when the piston tube 20 moves forward, on a front portion thereof, and a connecting tube portion 25, in which a fluid tube 100 is inserted to be connected, on a rear portion thereof In addition, the piston tube 20 includes a separation prevention protrusion 23, which is formed as a hollow tube shape to be blocked by the escape prevention portion 13 so as not to separate from the cylinder tube 10 when the piston tube 20 is retrieved to an outermost portion, on an outer surface thereof That is, the piston tube 20 is an element moving back and forth selectively in the cylinder tube 10 according to a manipulation power of the operator. As shown in the drawings, the piston tube 20 is a cylindrical member formed of a hard or soft material having a predetermined length and a hollow inner portion. The rear end portion of the piston tube 20 is opened so that the connecting tube portion 25, in which an end of the fluid tube 100 is inserted, is formed, and the separation prevention protrusion 23, which is blocked by the escape prevention portion 13, protrudes from the outer surface of the rear end portion.

The gasket 27 is integrally formed with an end portion of the piston tube 20 so that an outer surface of the gasket 27 is adhered to the inner surface of the cylinder tube 10 in order to prevent the solution from leaking out of the cylinder tube 10. The gasket 27 applies a suction pressure or a compressive pressure to a space between the front inner portion of the cylinder tube 10 and the piston tube 20, and at the same time, prevents the injection solution such as the blood or the aqueous solution from leaking between the cylinder tube 10 and the piston tube 20.

The reversible disposable resin needle for a blood vessel according to the present invention has a configuration similar to that of the conventional reversible disposable resin needle for a blood vessel filed by the present applicant. However, the cylinder tube 10 of the present embodiment is much shorter than that of the conventional art filed by the present applicant, and thus, the cylinder tube, that is, a needle, or the metallic needle, can be prevented from falling out of the blood vessel even when the patient moves his or her joints or body, and additionally, leakage of the injection solution is stably prevented. Accordingly, the injection can be performed stably while handling the syringe easily. To do this, the length of the piston tube 20 of the present embodiment is shorter than the entire length of the cylinder tube 10, and the resin tube 11 also has an appropriately short length. For example, the length of the cylinder tube 10 is reduced as much as possible provided that there is no limitation in using the resin needle, and the length of the piston tube 20 is shorter than that of the cylinder tube 10. In addition, a gap between the inner surface of the resin tube 11 and an outer surface of the metallic needle 21 is formed to be the smallest so that they may slide relative to each other while close contacting with each other. In addition, the metallic needle 21 is formed to have an inner diameter that is wider than that of any other metallic needles so that most of the solution may pass through the metallic needle 21. That is, the reversible disposable resin needle 1 for a blood vessel according to the present embodiment is configured so that the piston tube 20 has a length of about 60 to 90% based on the entire length of the cylinder tube 10 and the resin tube 11 has a length of about 40 to 60 mm. In addition, the gap between the inner surface of the resin tube 11 and the outer surface of the metallic needle 21 is about 0.05 to 1 mm. For example, if the cylinder tube 10 has a length of about 50 mm, the piston tube 20 is formed to have a length of about 30 to 40 mm. In addition, if the resin tube 11 of the cylinder tube 10 has a diameter of about 2 mm, the metallic needle 21 has a diameter of about 1 to 1.95 mm so that most of the solution passes through the hollow metallic needle 21.

An example of using the reversible disposable resin needle for a blood vessel according to the present embodiment will be described below. In a solution set including a container for storing blood or an aqueous solution (hereinafter referred to as a solution), in a state in which the connecting tube portion 25 of the piston tube 20 is connected to the fluid tube having an end connected to the container, the piston tube 20 is moved forward to a front maximum location in the cylinder tube 10. Then, the metallic needle 21 of the piston tube 20 is completely inserted into the resin tube 11 of the cylinder tube 10, and then the leading edge of the metallic needle 21 protrudes about 1 to 2 mm from the resin tube 11.

In the above state, when the operator inserts the metallic needle 21 into a blood vessel by holding the cylinder tube 10, the blood in the blood vessel flows back along the metallic needle 21 into the cylinder tube 10, and thus the medical operator may identify precisely whether the metallic needle 21 is inserted into the blood vessel. Here, when it is identified that the metallic needle 21 is accurately inserted into the blood vessel, the resin tube 11 of the cylinder tube 10 is also located in the blood vessel with the metallic needle 21. Thus, when the piston tube 20 including the metallic needle 21 is moved to a retrieving location within the cylinder tube 10, the injection operation is smoothly performed. Also, if the blood vessel is not found accurately at first, the injection can be tried repeatedly on the same person in a state in which the leading edge of the metallic needle 21 is exposed from the resin tube 11 by moving the piston tube 20 including the metallic needle 21 forward. FIGS. 7 through 12 show a reversible disposable resin needle for a blood vessel according to a second embodiment of the present invention. FIG. 7 shows a state in which the piston tube 20 moves forward in the cylinder tube 10 until the metallic needle 21 is exposed through the leading edge of the resin tube 11, FIG. 8 shows a state in which the piston tube 20 of FIG. 7 is retrieved, and FIG. 9 is a perspective view showing the cylinder tube 10 and the piston tube 20 separated from each other. FIGS. 10 and 11 are cross-sectional views for describing an inner configuration of the reversible disposable resin needle for a blood vessel shown in FIG. 7, and FIG. 12 is a cross-sectional view showing a state in which a fluid tube is connected to the reversible disposable resin needle for a blood vessel shown in FIG. 7. As shown in the above drawings, the reversible disposable resin needle 1 for a blood vessel according to the present embodiment is connected to the fluid tube 100 of a well known solution set (not shown) for injecting blood or an injection solution such as an aqueous solution into a blood vessel. The reversible disposable resin needle 1 for a blood vessel includes the cylinder tube 10 formed of a flexible and transparent material, the piston tube 20 configured to be inserted into the cylinder tube 10 to slidably move back and forth selectively according to an operation of an operator, and a film cylinder 30 for sealing the leading edge of the piston tube 20 and an inside of the cylinder tube 10 from each other.

The cylinder tube 10 includes the resin tube 11 having a hollow inside and a sharp end for injecting the injection solution into the blood vessel, and the body 12 integrally formed as a hollow cylinder on a rear portion of the resin tube 11. In addition, the escape prevention portion 13 for preventing the piston tube 20 that will be described later from escaping from the cylinder tube 10 is formed on a rear inner surface of the body 12.

The piston tube 20 is a member moving back and forth in the cylinder tube 10. The piston tube 20 includes the metallic needle 21, which has a hollow inner portion and is partially exposed through the leading edge of the resin tube 11 when the piston tube 20 moves forward, and a solution transfer path 22 formed at a side of the metallic needle 21 so that the injection solution is supplied into the cylinder tube 10. The connecting tube portion 25 including the gasket 27 that is adhered to the inner surface of the cylinder tube 10 and in which the fluid tube 100 is inserted is integrally disposed on a rear end portion of the solution transfer path 22. In addition, the separation prevention protrusion 23 that is projected by the escape prevention portion 13 so as not to be separated from the cylinder tube 10 when the piston tube 20 is retrieved to the maximum is formed on a rear outer surface of the connecting tube portion 25.

The film cylinder 30 is a cylindrical sealing element, opposite ends of which are connected to a rear inner surface of the body 12 of the cylinder tube 10 and an outer end surface of the connecting tube portion 25 located at the rear end of the gasket 27 of the piston tube 20. That is, the film cylinder 30 blocks leakage of the injection solution such as the aqueous solution or the blood through the space between the cylinder tube 10 and the piston tube 20, and is formed of a thin film of the tubular shape to form a cylindrical shape.

The reversible disposable resin needle 1 for a blood vessel according to the present embodiment has a configuration similar to that of the previous embodiment. However, the piston tube 20 of the present embodiment is configured to be shorter than the entire length of the cylinder tube 10 and the resin tube 11 is also formed to have an appropriate short length, and the gap between the inner surface of the resin tube 11 and the outer surface of the metallic needle 21 is wider than that of a conventional resin needle so that a flow amount of the solution can be increased. That is, the piston tube 20 according to the present embodiment has a length of about 60 to 90% based on the length of the cylinder tube 10 and the resin tube 11 has a length of about 40 to 60 mm. In addition, the gap between the inner surface of the resin tube 11 and the outer surface of the metallic needle 21 is about 0.5 to 2 mm. Through the above configuration, in the reversible disposable resin needle 1 for a blood vessel according to the present embodiment, the solution transfer path 22 is formed in the piston tube 20, and the gap between the inner surface of the resin tube 11 and the outer surface of the metallic needle 21 is wider than that of the previous embodiment. Thus, a large amount of solution flows through the gap between the metallic needle 21 and the resin tube 11. Also, since the film cylinder 30 is additionally formed in addition to the gasket 27 that is the sealing member, the leakage of the injection solution, a risk of bacterial infection, and infiltration of impurities can be completely blocked. Operations of the reversible disposable resin needle for a blood vessel according to the second embodiment of the present invention are the same as those of the previous embodiment, and thus detailed descriptions are omitted.

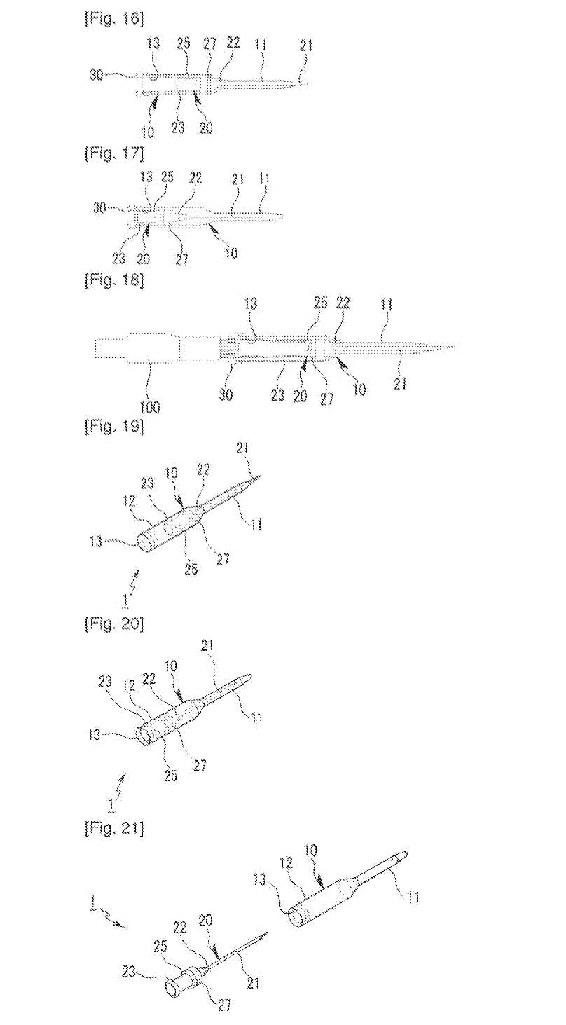

FIGS. 13 through 18 are diagrams showing a reversible disposable resin needle for a blood vessel according to a third embodiment of the present invention. FIG. 13 shows a state in which the piston tube 20 moves forward in the cylinder tube 10 until the metallic needle 21 is exposed through the leading edge of the resin tube 11, FIG. 14 shows a state in which the piston tube 20 of FIG. 13 is retrieved, and FIG. 15 is an exploded perspective view showing the cylinder tube 10 and the piston tube 20 separated from each other. FIGS. 16 and 17 are cross-sectional views for describing an inner configuration of the reversible disposable resin needle for a blood vessel shown in FIG. 13, and FIG. 18 is a cross-sectional view showing a state in which a fluid tube is connected to the reversible disposable resin needle for a blood vessel shown in FIG. 13. As shown in the drawings, the resin needle 1 of the present embodiment includes the cylinder tube 10, the piston tube 20, and the film cylinder 30.

The cylinder tube 10 includes the resin tube 11 having a hollow inside and a sharp end for injecting the injection solution into the blood vessel, and the body 12 formed as a hollow cylinder, in which the escape prevention portion 13 is formed on a rear inner surface, on a rear portion of the resin tube 11.

The piston tube 20 is a member moving back and forth in the cylinder tube 10. The piston tube 20 includes the metallic needle 21, which has a hollow inner portion and is partially exposed through the leading edge of the resin tube 11 when the piston tube 20 moves forward, and a solution transfer path 22 formed at a side of the metallic needle 21 so that the injection solution is supplied into the cylinder tube 10. The connecting tube portion 25 including the gasket 27 that is adhered to the inner surface of the cylinder tube 10 and in which the fluid tube 100 is inserted is disposed on a rear end portion of the solution transfer path 22. In addition, the separation prevention protrusion 23 that is projected by the escape prevention portion 13 so as not to be separated from the cylinder tube 10 when the piston tube 20 is retrieved to the maximum is formed on a rear outer surface of the connecting tube portion 25.

The film cylinder 30 is a cylindrical sealing element, opposite ends of which are connected to a rear inner surface of the body 12 of the cylinder tube 10 and an outer end surface of the connecting tube portion 25 of the piston tube 20. The film cylinder 30 blocks leakage of the injection solution such as the aqueous solution or the blood through the space between the cylinder tube 10 and the piston tube 20, and is formed of a thin film of a tubular shape to form a cylindrical shape so as to completely prevent a risk of bacterial infection between inner and outer portions and the infiltration of impurities.

Meanwhile, the piston tube 20 of the present embodiment is configured to be shorter than the entire length of the cylinder tube 10 and the resin tube 11 is also formed to have an appropriately short length, and the gap between the inner surface of the resin tube 11 and the outer surface of the metallic needle 21 is wider than that of a conventional resin needle. That is, the piston tube 20 has a length of about 60 to 90% based on the entire length of the cylinder tube 10 and the resin tube 11 has a length of about 40 to 60 mm. In addition, the gap between the inner surface of the resin tube 11 and the outer surface of the metallic needle 21 is formed to have about 0.5 to 2 mm. The reversible disposable resin needle 1 for a blood vessel according to the third embodiment of the present invention has a configuration similar to that of the second embodiment, except for the connecting location of the film cylinder 30.

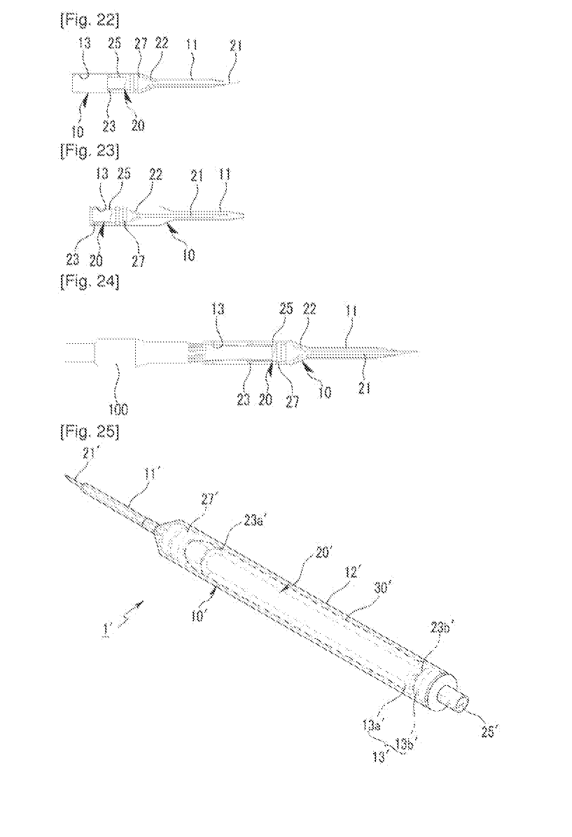

FIGS. 19 through 24 are diagrams showing a reversible disposable resin needle for a blood vessel according to a fourth embodiment of the present invention. FIG. 19 shows a state in which the piston tube 20 moves forward in the cylinder tube 10 until the metallic needle 21 is exposed through the leading edge of the resin tube 11, FIG. 20 shows a state in which the piston tube 20 of FIG. 19 is retrieved, and FIG. 21 is an exploded perspective view showing the cylinder tube 10 and the piston tube 20 separated from each other. FIGS. 22 and 23 are cross-sectional views for describing an inner configuration of the reversible disposable resin needle for a blood vessel shown in FIG. 19, and FIG. 24 is a cross-sectional view showing a state in which a fluid tube is connected to the reversible disposable resin needle for a blood vessel shown in FIG. 19. As shown in the drawings, the resin needle 1 of the fourth embodiment of the present invention includes the cylinder tube 10, the piston tube 20, and the gasket 27.

The cylinder tube 10 includes the resin tube 11 having a hollow inside and a sharp end for injecting the injection solution into the blood vessel, and the body 12, in which the escape prevention portion 13 is formed on a rear inner surface, connected integrally to a rear portion of the resin tube 11.

The piston tube 20 is a member moving back and forth in the cylinder tube 10. The piston tube 20 includes the metallic needle 21, which has a hollow inner portion and is partially exposed through the leading edge of the resin tube 11 when the piston tube 20 moves forward, and the solution transfer path 22 formed at a side of the metallic needle 21 so that the injection solution is supplied into the cylinder tube 10. In addition, the separation prevention protrusion 23, in which the fluid tube 100 is inserted and which is projected by the escape prevention portion 13 so as not to be separated from the cylinder tube 10 when the piston tube 20 is retrieved to the maximum, is formed on a rear outer surface of the connecting tube portion 25.

The gasket 27 is a watertight element that is integrally disposed on an end of the connecting tube portion 25 of the piston tube 20, and an outer surface of the gasket 27 is in slidable contact with the inner surface of the cylinder tube 10 and in close contact with the inner surface of the cylinder tube 10. The gasket 27 can be embodied by well known art. Meanwhile, the piston tube 20 has a length of about 60 to 90% based on the entire length of the cylinder tube 10 and the resin tube 11 has a length of about 40 to 60 mm. In addition, the gap between the inner surface of the resin tube 11 and the outer surface of the metallic needle 21 is about 0.5 to 2 mm. The reversible disposable resin needle for a blood vessel according to the fourth embodiment of the present invention has a configuration similar to that of the first embodiment, except that the solution transfer path 22 is formed and the gap between the resin tube 11 and the metallic needle 21 is increased so that a large amount of injection solution can flow.

As described above, the present invention is provided to address the problems of the conventional resin needle having a relatively longer length filed by the present applicant, that is, behavior of the patient is restricted, and thus the patient may feel inconvenience, and moreover, if the needle falls out of the blood vessel due to the movements of arms or joints of the patient, the injection operation has to be performed again using a new needle. That is, the length is reduced less than the conventional syringe, that is, the cylinder tube forming the outer body is formed to have a length of about 40 to 60 mm, and the gap between the resin tube and the metallic needle is regulated in order to ensure a sufficient flow of the injection solution. Thus, convenience of the handling and storage can be improved, and in particular, the patient may freely move his or her arms or joints when the injection is performed.

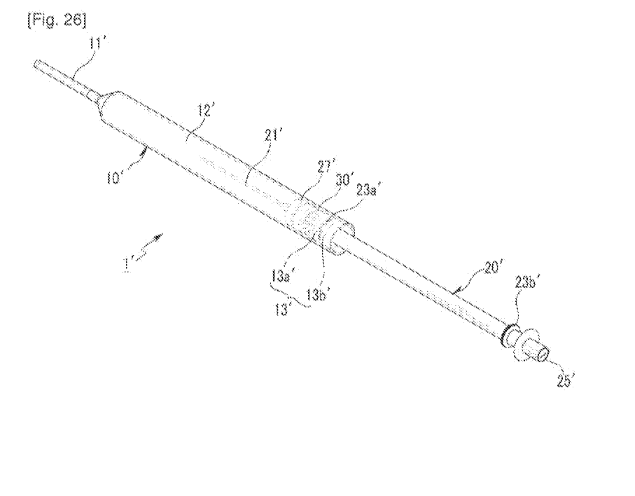

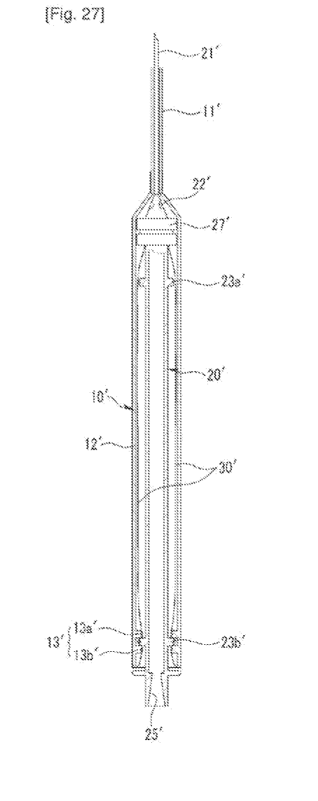

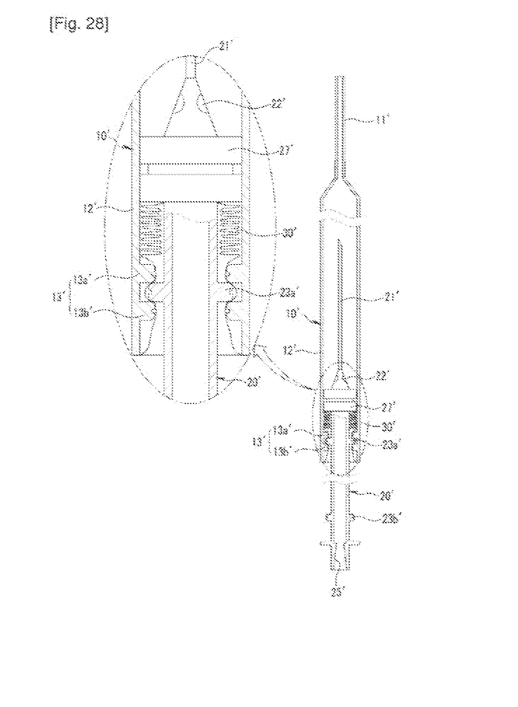

FIGS. 25 through 28 are diagrams showing a reversible disposable resin needle for a blood vessel according to a fifth embodiment of the present invention. Referring to the drawings, the resin needle of the present embodiment includes a cylinder tube 10′ formed of a flexible and transparent material, a piston tube 20′ configured to be inserted in the cylinder tube 10′ to slidably move back and forth selectively according to an operation of an operator, and a film cylinder 30′ formed of a cylindrical film connecting the piston tube 20′ and the cylinder tube 10′ to each other.

The cylinder tube 10′ is a cylindrical member having a tubular shape formed of a transparent or semitransparent and hard or flexible material so that an operator may recognize the inside of the cylinder tube 10′, and has a leading edge on which a resin tube 11′ integrally protrudes and an opened rear end. Also, a fixed suspension member 13′ for forming a projection recess is integrally formed with a rear inner surface of the cylinder tube 10′. The fixed suspension member 13′ is formed of a pair of fixed suspension projections 13a and 13b′ protruding in front and rear portions in a length direction of the cylinder tube 10′, and a gap between the pair of fixed suspension projections 13a and 13b′ forms the projection recess in which restriction projections 23a′ and 23b′ formed on front and rear outer surfaces of the piston tube 20′, which will be described below, are projected. The fixed suspension member 13′ may preferably protrude to a predetermined height so as not to come in contact with the outer surface of the piston tube 20′, that is, so that position interference does not occur.

The piston tube 20′ is included in the cylinder tube 10′ to be selectively moved back and forth by the manipulation of the operator, and is formed as a hollow cylinder having a predetermined length formed of a hard or soft material. A rear end portion of the piston tube 20′ is opened, on which a connecting tube 25′ connecting to a fluid tube is formed, and a location opposite to the connecting tube 25′, that is, a leading edge, includes one or a plurality of solution transfer paths 22′ that are penetration holes through which the solution or the blood induced from the rear end portion can flow toward the cylinder tube 10′. Also, the piston tube 20′ includes a gasket 27′ having an extended diameter so as to be in slidable contact with an inner circumferential surface of the cylinder tube 10′ at a rear portion of the solution transfer paths 22′, and the gasket 27′ applies a suction pressure or a compressive pressure to a space between the solution transfer paths 22′ and the inside of the leading edge of the cylinder tube 10′, and at the same time, prevents leakage of the injection solution such as the blood or the aqueous solution between the cylinder tube 10′ and the piston tube 20′. Also, the piston tube 20′ includes a metallic needle 21′ having a predetermined length toward the solution transfer paths 22′, and the metallic needle 21′ is configured to be inserted into the resin tube 11′. According to the piston tube 20′ having the above configuration, a partial leading edge of the metallic needle 21′ is exposed in the resin tube 11′ when the piston tube 20′ is moved forward to the maximum in the cylinder tube 10′.

The film cylinder 30′ is a sealing element for preventing leakage of the injection solution such as the aqueous solution or the blood through the space between the cylinder tube 10′ and the piston tube 20′. The film cylinder 30′ is formed of a thin film in a tubular shape, and is configured not to be exposed to the outside when the piston tube 20′ is retrieved to the maximum within a cylinder 10s′ without being separated. To do this, a leading edge of the film cylinder 30′ is bonded to a side adjacent to the gasket 27′ of the piston tube 20′, and a rear end of the film cylinder 30′ is bonded to a rear inner surface of the cylinder tube 10′ so as to be folded or unfolded in connection with the back and forth movement of the piston tube 20′ in the space between the cylinder tube 10′ and the piston tube 20′. That is, the film cylinder 30′ is formed to surround a partial outer surface of the piston tube 20′, and a side of the film cylinder 30′ is bonded to a side of the gasket 27′ and the other side of the film cylinder 30′ is connected to a side of the fixed suspension member 13′ that protrudes from the rear inner surface of the cylinder tube 10′.

Meanwhile, the cylinder tube 10′ and the piston tube 20′ further include a location determination element for stably maintaining a location of the piston tube 20′ when the piston tube 20′ moves back and forth. The location determination element may be realized by forming projection type members protruding respectively from the inner circumferential surface of the cylinder tube 10′ and an outer circumferential surface of the piston tube 20′ so as to be relatively projected by each other. The location determination element may include a retrieval restriction projection 23a′ and a proceed restriction projection 23b′ protruding from front and rear outer surfaces of the piston tube 20′, and the fixed suspension member 13′ having a projection recess by which the restriction projections 23a′ and 23b′ are selectively projected on the rear inner surface of the cylinder tube 10′. That is, the location determination element forms the proceed restriction projection 23b′ on the rear outer surface of the piston tube 20′ so as to restrict proceeding movement of the piston tube 20′, and forms the fixed suspension member 13′ forming the projection recess on the rear inner surface of the corresponding cylinder tube 10′ so that the proceed restriction projection 23b′ can be projected when the operator manipulates the piston tube 20′ in a state in which the piston tube 20′ is located at the frontmost portion within the cylinder tube 10′. Also, the location determination element forms the retrieval restriction projection 23a′ on the front outer surface of the piston tube 20′ so as to restrict retrieving movement of the piston tube 20′ when the operator manipulates the piston tube 20′ in a state in which the piston tube 20′ is located at the rearmost portion within the cylinder tube 10′. The retrieval restriction projection 23a′ is formed at a location at which the retrieval restriction projection 23a′ is projected by the projection recess of the fixed suspension member 13′ when the piston tube 20′ retrieves to the maximum in the cylinder tube 10′.

An example of using the reversible disposable resin needle for a blood vessel according to the present embodiment will be described below. In a state in which the connecting tube 25′ of the piston tube 20′ is connected to the fluid tube, the piston tube 20′ is moved forward to the frontmost position within the cylinder tube 20′. Then, the metallic needle 21′ of the piston tube 20′ is completely inserted in the resin tube 11′ of the cylinder tube 10′ and the leading edge of the metallic needle 21′ protrudes about 1 to 2 mm from the resin tube 11′. In the above state, when the metallic needle 21′ is inserted into the blood vessel on which injection is to be performed by holding the cylinder tube 10′, the blood in the blood vessel flows back into the metallic needle 21′ toward the cylinder tube 10′ so that the medical operator may identify exactly whether the metallic needle 21′ is inserted into the blood vessel. Here, if it is identified that the metallic needle 21′ is accurately inserted into the blood vessel, the resin tube 11′ of the cylinder tube 10′ is also located in the blood vessel with the metallic needle 21′, and thus the injection can be performed by moving the piston tube 20′ to the retrieval location in the cylinder tube 10′.

1. A reversible disposable resin needle for a blood vessel, the resin needle comprising:
a cylinder tube (10) including a resin tube (11) having a hollow inner portion to inject an injection solution into a blood vessel and a body (12) integrally connected to a rear end of the resin tube (11) and including an escape prevention portion (13) on a rear inner surface;
a piston tube (20) formed as a hollow cylinder moving back and forth in the cylinder tube (10), wherein the piston tube comprises a metallic needle (21) having a hollow inside and protruding from a leading edge of the piston tube to be partially exposed from a leading edge of the resin tube (11) when the piston tube proceeds, a connecting tube (25) to which a fluid tube (100) is inserted on a rear end of the piston tube, and a separation prevention projection (23) projected by the escape prevention portion (13) on an outer surface of the piston tube when the piston tube is retrieved to a maximum in the cylinder tube (10); and
a gasket (27) that is a watertight element disposed integrally with an end of the piston tube (20) and having an outer surface in slidable close contact with an inner surface of the cylinder tube (10),
wherein the piston tube (20) has a length of about 60 to 90% based on an entire length of the cylinder tube (10), the resin tube (11) has a length of about 40 to 60 mm, and a gap between the inner surface of the resin tube (11) and an outer surface of the metallic needle (21) is about 0.05 to 1 mm.

2. A reversible disposable resin needle for a blood vessel, the resin needle comprising:
a cylinder tube (10) including a resin tube (11) having a hollow inner portion and a sharp end to inject an injection solution into a blood vessel and a body (12) integrally connected to a rear end of the resin tube (11) and including an escape prevention portion (13) on a rear inner surface;
a piston tube (20) formed as a hollow cylinder moving back and forth in the cylinder tube (10), wherein the piston tube (20) comprises a metallic needle (21) having a hollow inside and protruding from a leading edge of the piston tube (20) to be partially exposed from a leading edge of the resin tube (11) when the piston tube (20) proceeds, a solution transfer path (22) formed at a side of the metallic needle (21) and configured to supply an injection solution into the cylinder tube (10), and a connecting tube (25) to which a fluid tube (100) is inserted on a rear end of the piston tube (20) and having a gasket (27) in close contact with the inner surface of the cylinder tube (10) on an outer surface of the piston tube (20), and a separation prevention projection (23) projected by the escape prevention portion (13) on an outer surface of the piston tube (20) when the piston tube (20) is retrieved to a maximum in the cylinder tube (10); and
a film cylinder (30) that is a sealing element of a cylindrical shape having opposite ends connected respectively to a rear inner surface of the body (12) of the cylinder tube (10) and the connecting tube (25) at a rear end of the gasket (27) of the piston tube (20),
wherein the piston tube (20) has a length of about 60 to 90% based on an entire length of the cylinder tube (10), the resin tube (11) has a length of about 40 to 60 mm, and a gap between the inner surface of the resin tube (11) and an outer surface of the metallic needle (21) is about 0.5 to 2 mm.

3. A reversible disposable resin needle for a blood vessel, the resin needle comprising:
a cylinder tube (10) including a resin tube (11) having a hollow inner portion and a sharp end to inject an injection solution into a blood vessel and a body (12) integrally connected to a rear end of the resin tube (11) and including an escape prevention portion (13) on a rear inner surface;
a piston tube (20) formed as a hollow cylinder moving back and forth in the cylinder tube (10), wherein the piston tube (20) comprises a metallic needle (21) having a hollow inside and protruding from a leading edge of the piston tube (20) to be partially exposed from a leading edge of the resin tube (11) when the piston tube (20) proceeds, a solution transfer path (22) formed at a side of the metallic needle (21) and configured to supply an injection solution into the cylinder tube (10), and a connecting tube (25) to which a fluid tube (100) is inserted on a rear end of the piston tube (20) and having a gasket (27) in close contact with the inner surface of the cylinder tube (10) on an outer surface of the piston tube (20), and a separation prevention projection (23) projected by the escape prevention portion (13) on an outer surface of the piston tube (20) when the piston tube (20) is retrieved to a maximum in the cylinder tube (10); and
a film cylinder (30) that is a sealing element of a cylindrical shape having opposite ends connected respectively to a rear inner surface of the body (12) of the cylinder tube (10) and the connecting tube (25) at a rear end of the gasket (27) of the piston tube (20),
wherein the piston tube (20) has a length of about 60 to 90% based on an entire length of the cylinder tube (10), the resin tube (11) has a length of about 40 to 60 mm, and a gap between the inner surface of the resin tube (11) and an outer surface of the metallic needle (21) is about 0.5 to 2 mm.

4. A reversible disposable resin needle for a blood vessel, the resin needle comprising:
a cylinder tube (10) including a resin tube (11) having a hollow inner portion and a sharp end portion to inject an injection solution into a blood vessel and a body (12) integrally connected to a rear end of the resin tube (11) and including an escape prevention portion (13) on a rear inner surface;
a piston tube (20) formed as a hollow cylinder moving back and forth in the cylinder tube (10), wherein the piston tube (20) comprises a metallic needle (21) having a hollow inside and protruding from a leading edge of the piston tube (20) to be partially exposed from a leading edge of the resin tube (11) when the piston tube (20) proceeds, a solution transfer path (22) formed at a side of the metallic needle (21) and configured to supply an injection solution into the cylinder tube (10), and a separation prevention projection (23), in which a fluid tube (100) is inserted, projected by the escape prevention portion (13) on an outer surface of the piston tube (20) when the piston tube (20) is retrieved to a maximum in the cylinder tube (10); and
a gasket (27) that is a watertight element disposed integrally with an end of the connecting tube (25) of the piston tube (20) and having an outer surface in slidable contact with an inner surface of the cylinder tube (10),
wherein the piston tube (20) has a length of about 60 to 90% based on an entire length of the cylinder tube (10), the resin tube (11) has a length of about 40 to 60 mm, and a gap between the inner surface of the resin tube (11) and an outer surface of the metallic needle (21) is about 0.5 to 2 mm.

5. (canceled)
